(19) United States
(12) Patent Application Publication
(10) Pub. No.: US 2009/0054842 A1
(43) Pub. Date: Feb. 26, 2009

(54) Enhanced penetration system and method for sliding microneedles

(75) Inventors: Yehoshua Yeshurun, Haifa (IL); Meir Hefetz, Mizpeh Harashim (IL); Gil Fruchtman, Kiryat Tivon (IL); Yoel Sefi, Merom Hagalil (IL); Yotam Levin, Tel Aviv (IL); Gilad Lavi, Rishon Lezion (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: Nanopass Technologies Ltd., Haifa (IL)

(21) Appl. No.: 10/595,859

(22) PCT Filed: Nov. 18, 2004
(86) PCT No.: PCT/IL04/01065

§ 371 Date: Oct. 29, 2008

Related U.S. Application Data
(60) Provisional application No. 60/520,667, filed on Nov. 18, 2003.
(60) Provisional application No. 60/581,711, filed on Jun. 23, 2004.

(51) Int. Cl. A61M 5/32

(52) U.S. Cl. 604/173

(57) ABSTRACT

A microneedle device for transporting fluid through a surface of a biological barrier, the device including a fluid transport configuration, an abutment member and a displacement device. The fluid transport configuration includes a substrate having a substantially planar surface having a plurality of microneedles projecting therefrom. The abutment member has an abutment surface for abutting the biological barrier. The displacement device is configured for generating a relative lateral sliding movement between the surface of the biological barrier and the fluid transport configuration in a sliding direction of the microneedles The microneedles are arranged so that a leading microneedle defines an effective area which is effective void of another microneedle. The effective area is defined as an area marked out by translating the base area of the leading microneedle, by the height of the leading microneedle, in a direction opposite to the sliding direction.

### FIELD AND BACKGROUND OF THE INVENTION

The present invention relates to microneedles and, in particular, it concerns an enhanced penetration system and method for sliding microneedles.

Research and development of microneedle arrays has advanced in recent years as part of a system for drug delivery or biological sampling. In these applications, the microneedle approach shows clear advantages over competing methods of transferring fluids through skin or other barriers. In contrast to hypodermic needles, microneedles are painless, allowing shallow delivery to the epidermis. Unlike many needle applications, microneedle systems can be self administered or administered by non professionals. Additionally, the potential risk of accidental needle jabs and related injuries is largely avoided. In addition, microneedle based devices overcome the molecular size limitations characteristic of conventional transdermal patches, which are inherently limited to small molecules (less than 1,000 dalton and typically less than 300 dalton). Furthermore, unlike other delivery systems that incorporate an active, usually energy driven, hole forming mechanism (for example, ultrasound, RF or laser delivery first requires making holes in the skin and then applying a topical drug or patch), microneedles are able to combine the enhancement/penetration mechanism with the drug itself thereby allowing easy application of the drug. Examples of such work may be found in PCT Publications Nos. WO01/66065 and WO 02/17985, both co-assigned with the present application. These publications are hereby incorporated by reference as if set out in their entirety herein. Other relevant publications include WO 99/64580 and WO 00/74763 to Georgia Tech Research Corp., as well as in the following scientific publications: “Micro machined needles for the transdermal delivery of drugs”, S. H. S. Henry et al. (MMS 98, Heildelberg, Germany, Jan. 1998); “Three dimensional hollow micro needle and microtube arrays”, D. V. McAllister et al. (Transducer 99, Sendai, Japan, June 1999); “An array of hollow micro-capillaries for the controlled injection of genetic materials into animal/plant cells”, K. Chun et al. (MEMS 99, Orlando, Fl., January 1999); and “Injection of DNA into plant and animal tissues with micromechanical piercing structures”, W. Trimmer et al. (IEEE workshop on MEMS, Amsterdam, January 1995). The aforementioned PCT applications disclose the use of hollow microneedles to provide a flow path for fluid flow through the skin barrier.

While hollow microneedles are potentially an effective structure for transferring fluids across a biological barrier, the devices proposed to date suffer from a number of drawbacks that limit or prevent their functionality.

Current microneedle array devices do not reliably penetrate the biological barrier, preventing or diminishing cross-barrier transfer of fluids. In the case of administering drugs through human skin, the transfer is ineffective if the microneedle does not pierce at least the stratum corneum layer. In many cases, the skin surface is elastic enough to stretch around each microneedle without being pierced. Lack of sharpness of many microneedles exasperates this phenomenon. Additionally, the fragility, especially under sheer forces, of various microneedle designs limit the penetration force applied to the microneedles, thereby limiting penetration efficacy. Further, many microneedle designs include truncated microneedles. Truncation results in both clogging of the needle channels, and a reduction of sharpness of the needle, again leading to poor penetration and poor material delivery.

Various approaches have been proposed to ensure sufficient penetration into the skin One approach has been to use very long and sharp microneedles. While achieving greater penetration, the microneedles produced by this method are more fragile and more difficult to manufacture. A different approach is suggested by the aforementioned WO 00/74763 to Georgia Tech which proposes various complicated mechanical devices to stretching the skin. U.S. Pat. No. 6,440,096 to Lastovish et al. discloses an arrangement for stretching the skin by use of a suction cup constructed around the device. Yet another approach is based on diminishing the elasticity of the skin by freezing or otherwise changing the mechanical properties of the skin prior to penetration. All of these approaches clearly suffer from complexity of use, and/or production, cost issues and potential lack of patient compliance.

In the field of surgical tools for use during surgical procedures, it is known to use ultrasonic vibrations to enhance the effect of a cutting or separating tool as in U.S. Pat. No. 4,832,683 to Idemoto et al. Ultrasonic vibrations have been a feature of surgical devices intended for use by skilled personnel, but have not been previously applied to enhance penetration of microneedles into a biological barrier.

It is also known to employ a needleless injector as an alternative to a hollow needle for injection of fluid into the body. These injectors use a fine stream or “jet” of pressurized liquid to penetrate the skin. Early designs used high pressure throughout the injection, to punch a hole through the tough stratum corneum and epidermis.

However, the bulk of the injection could then be infused along the initial track under much lower pressure. U.S. Pat. No. 2,704,542 to Scherer and U.S. Pat. No. 3,908,651 to Fudge disclose examples of this design. Ultimately, the engineering demands of changing the pressure during the injection and resulting complexity, the cost, and the pain associated, have limited the use of such devices.

In some cases, modern high-pressure needleless jet injectors are driven by pressure from a pressurized gas cylinder as exemplified by U.S. Pat. Nos. 6,063,053 and 6,264,629. U.S. Pat. No. 5,499,972 teaches a jet injection device powered by a powerful cocked spring. Of most relevance to the present invention are U.S. Pat. Nos. 6,102,896 and 6,224,567 which teach a jet injection device where the pressure is generated manually by pressing on a cap. When sufficient force is applied, a mechanical obstruction is overcome to actuate the pressure jet. While jet injectors offer advantages of somewhat reduced pain and potentially improved hygiene compared to conventional needle injections, they still suffer from many drawbacks. Jet injection depends on a specific positioning of the device relative to the site, and any slight change in that position can end with drug loss or risk of wound (“wet injection”). Two more constraints are high sheer forces applied on the molecules thereby requiring specific validation for each formulation and use of non-standard drug cartridges. Most notably, since there is no sealed conduit between the drug supply and the target tissue, significant wastage of the drug occurs. This also results in lack of precision in the administered dosage of a drug. Furthermore, penetration through the strong tissue of the upper layers of the skin requires high activation pressures which typically require complex and expensive systems. The use of purely manual pressure for activation may raise questions of reliability. Finally, most injectors penetrate to the deep subcutaneous and muscle layers and are incapable of shallow, consistent, delivery in the epidermis or shallow dermis. This may limit their applicability to applications using those locations, for example during vaccination delivery.

WO 03/074102, co-assigned with the present application, which is incorporated by reference for all purposes as if fully set forth herein, teaches improved microneedle penetration devices. The device of the aforementioned publication uses directional insertion, preferably using asymmetric microneedles, such as micropyramids (pyramid shaped microneedles with cutting edges or blades), to enhance penetration of the biological barrier. It is explained in the aforementioned publication that the flexibility of the skin is thought to be pronounced under out-of-plane deformations, allowing the skin to be locally depressed so as to conform to the external shape of the microneedles without allowing proper penetration. This effect seriously impedes, or even prevents, fluid transfer via the microneedles. However, directional insertion device of the aforementioned publication includes generating a displacement of the microneedle substrate relative to the biological barrier, the displacement having a non-zero component parallel to the surface of the substrate. In contrast to the out-of-plane flexibility of the biological barrier, the in-plane stretching capabilities of the skin are much more limited. These contrasting properties are familiar to us from everyday experience in which relatively blunt objects which do not pierce the skin on localized pressure readily cause scratches under sliding contact conditions. As a result of these properties, a penetration vector which includes a component parallel to the skin surface tends to be much more effective than direct pressure perpendicular to the skin. It is also possible to anchor the skin against in-plane movement around the microneedle insertion region, thereby further enhancing the sliding penetration effect.

In particular, WO 03/074102 teaches improved devices using “sliding” asymmetrical microneedles having a cutting edge. Reference is now made to FIGS. 1a and 1b. FIG. 1a is an isometric view of a microneedle 10 that is constructed and operable in accordance with the prior art. FIG. 1b is another isometric view of microneedle 10 of FIG. 1a. Microneedle 10 has a penetrating tip 12, a cutting edge 14 and a channel 16 therein. Microneedle 10 is robust, has very thick walls and has a small aspect ratio. Microneedle 10 typically has a height of between 100 and 750 microns, a hole diameter of between 25 and 65 microns and a wall thickness between 20 and 75 microns. Penetrating tip 12 is extremely sharp. Cutting edge 14 enhances penetration of the microneedle by cutting the skin thereby reducing the surface tension of the skin which normally tends to push a microneedle out of the skid. Microneedle 10 is an example of a pyramidal microneedle, generally referred to as a micropyramid. Another example of a pyramidal microneedle is described below, describing the geometry and other advantages of the microneedle in more detail. A tubular microneedle example is also described below.

Reference is now made to FIGS. 2a-3c. FIG. 2a is a schematic isometric view of a pyramidal microneedle 18 that is constructed and operable in accordance with the prior art FIG. 2b is a schematic plan view of microneedle 18 of FIG. 2a. FIG. 2c is a schematic view of a base-to-tip vector 20 of microneedle 18 of FIG. 2a FIG. 3a is a schematic view tubular microneedle 22 that is constructed and operable in accordance with the prior art. FIG. 3b is a schematic plan view of microneedle 22 of FIG. 3a FIG. 3c is a schematic view of a base-to-tip vector 24 of microneedle 22 of FIG. 3a Microneedle 18 and microneedle 22 are asymmetrical such that base-to-tip vector 20 and base-to-tip vector 24, respectively, are non-perpendicular to a supporting surface 26 of a substrate 28. The directionality of the “base-to-tip vector” is then coordinated with the insertion path so as to enhance the penetration effect of the lateral (in-plane) displacement component. The calculation of the “base-to-tip vector” for microneedle 18 and microneedle 22 is illustrated graphically in FIGS. 2c and 3c, respectively. Geometrically, the “base-to-tip vector” is typically defined as a vector from a centroid of a base area of the microneedle to a centroid of a penetrating tip of the microneedle. In this context, the “centroid” of a shape is a point in the plane of a two-dimensional shape which, when used as an origin, the vector sum over the area of the shape is zero. In other words, the centroid corresponds to the center of mass of a thin slice of uniform weight per unit area corresponding to the shape of the cross-section. In the case of the microneedles of the present invention, the base centroid is the centroid of a cross-section of the microneedle form taken in the plane of surface 26 of substrate 28. Similarly, the tip centroid is the centroid of the area of a cross-section taken through the microneedle tip parallel to surface 26. In the case of a pointed microneedle, the tip centroid is effectively the sharp point itself. Microneedle 18 is a disclosed in the aforementioned PCT publication no. WO 02/17985, incorporated herein by reference. The base of microneedle 18 is substantially triangular such that the centroid falls somewhere near the intuitive “center” of the triangle. Microneedle 18 has a penetrating tip 30. Penetrating tip 30, on the other hand, is formed at the intersection of an inclined face with at least one substantially upright wall. As a result, the centroid of penetrating tip 30 is defined by the penetrating tip which is located roughly above one of the corners of the triangular base. The resulting base-to-tip vector 20 is illustrated in FIG. 2c and has a significant in-plane component. Parenthetically, it should be noted that the microneedle form of FIG. 2a is believed to be particularly advantageous for the mechanical support it provides to both the tip and the upright walls which make is highly suited to withstand the directional insertion without breakage. Furthermore, fluid transfer is greatly enhanced by use of a microneedle structure where a fluid transfer conduit intersects the microneedle surfaces at a position proximal to a solid penetrating tip, such as in this structure, thereby avoiding plugging of the conduit. Referring now to FIGS. 2b and 3b, there is illustrated a further alternative, or additional, preferred feature of the microneedle structure for directional insertion through a biological barrier. According to this feature, each microneedle is formed with at least two side walls 32 which form a relatively sharp edge 34 between them. Geometrically, a substantially planar face of each side wall is positioned such that an angle between the faces as measured in a plane parallel to the microneedle supporting base surface is no greater than 90 degrees, and preferably between 30 degrees and 70 degrees. It should be noted that the angle mentioned is defined between the substantially planar portions of the faces and does not exclude the possibility of rounding of the edge between the faces. This feature is effective in facilitating cutting of the biological barrier during directional insertion, even where the edge between the faces is somewhat rounded.

In all cases where this cutting-edge property is used, the direction of insertion is clearly chosen to have a component in the direction in which the cutting edge “points”, and specifically, such that the in-plane component of the insertion direction for at least part of the path of motion lies within the range of angles as illustrated in the plan views of FIGS. 2b and 3b.

Microneedles having cutting edges allow good penetration of the microneedles across a biological barrier. However, flexibility of the biological barrier tends to reduce penetration effectiveness even for microneedles having cutting edges, which are also known as micro blades.

There is therefore a need for a device and method for enhancing the penetration of a biological barrier, particularly the stratum corneum, by microneedles having cutting edges.

### SUMMARY OF THE INVENTION

The present invention is a microneedle device and method of operation thereof.

According to the teachings of the present invention there is provided, a microneedle device for transporting fluid through a surface of a biological barrier, the device comprising: (a) a fluid transport configuration including: (i) a substrate having a substantially planar surface; and (ii) a plurality of microneedles projecting from the planar surface, each of the microneedles having a cutting edge, a penetrating tip, a base area and a height; (b) an abutment member having at least one abutment surface for abutting the biological barrier, the abutment member being mechanically connected to the fluid transport configuration; and (c) a displacement device operationally connected to the abutment member, the displacement device configured for generating a relative lateral sliding movement between the surface of the biological barrier and the fluid transport configuration in a sliding direction of the microneedles, wherein the microneedles are arranged so that a leading one of the microneedles defines an effective area which is void of another of the microneedles, the effective area being defined as an area marked out by translating the base area of the leading microneedle, by the height of the leading microneedle, in a direction opposite to the sliding direction.

According to a further feature of the present invention, a spacing of the microneedles in the sliding direction is at least the square root of 2 times a closest neighbor spacing.

According to a further feature of the present invention: (a) the abutment member is configured as a suction cup, the fluid transport configuration being disposed in the suction cup; and (b) the displacement device includes a suction arrangement in fluid connection with the suction cup, the suction arrangement being configured for generating suction for pulling the surface of the biological barrier into the suction cup, the suction cup and the fluid transport configuration being configured such that the surface of the biological barrier slides across the planar surface in the sliding direction.

According to a further feature of the present invention: (a) the abutment surface lies on a first plane; (b) the surface of the substrate lies on a second plane; and (c) the first plane is oblique to the second plane.

According to a further feature of the present invention, the suction cup has an internal surface which is as asymmetrical.

According to a further feature of the present invention, the suction cup includes a side trough in fluid connection with the suction arrangement, the suction arrangement and the side trough being configured such that, after the surface of the biological barrier has made contact with the microneedles, the biological barrier is pulled into the side trough thereby pulling the surface of the biological barrier across the surface of the substrate.

According to a further feature of the present invention, the displacement device mechanically links the abutment member and the fluid transport configuration, the displacement device defining a path of movement of the fluid transport configuration relative to the abutment surface, at least part of the path of movement having a non-zero component parallel to the surface of the substrate.

According to a further feature of the present invention, the suction arrangement includes a suction plunger, the suction arrangement being configured for generating suction for pulling the surface of the biological barrier into the suction cup with a single one-directional movement of the suction plunger to a retracted position in the suction arrangement.

According to a further feature of the present invention, the suction arrangement includes a locking mechanism for retaining the suction plunger in the retracted position.

According to a further feature of the present invention, there is also provided a fluid injection plunger arrangement having a fluid plunger, the fluid injection plunger arrangement being in fluid connection with the fluid transport configuration, such that depressing the fluid plunger delivers the fluid via the fluid transport configuration.

According to a further feature of the present invention, the fluid injection plunger arrangement is disposed within the suction arrangement.

According to a further feature of the present invention, there is also provided a priming port in fluid connection with the fluid injection plunger arrangement, the priming port being configured for providing a fluid connection between an external supply of the fluid and the fluid injection plunger arrangement during filling of the fluid injection plunger arrangement with the fluid.

According to a further feature of the present invention, the fluid injection plunger arrangement has a movement restriction arrangement configured to prevent negative pressure within the suction cup from pulling down the fluid plunger.

According to a further feature of the present invention, at least one of the fluid transport configuration and the abutment member are configured such that, a leading one of the rows of the microneedles contacts the biological barrier prior to a trailing one of the rows of the microneedles contacting the biological barrier.

According to a further feature of the present invention, the displacement device is mechanically connected to the abutment member and the fluid transport configuration, the displacement device defining a rotational path of movement of the fluid transport configuration relative to the abutment member.

According to a further feature of the present invention, the rotational path of movement is about an axis substantially parallel to the initial orientation of the surface of the biological barrier.

According to the teachings of the present invention there is also provided a microneedle device for transporting fluid across a biological barrier, the device comprising: (a) a fluid transport configuration including: (i) a substrate having a substantially planar surface; and (ii) a plurality of microneedles projecting from the surface, each of the microneedles having a penetrating tip, a cutting edge, a base area and a height; (b) an abutment member having at least one abutment surface for abutting the biological barrier, and (c) a displacement device mechanically linking the abutment member and the fluid transport configuration, the displacement device defining a path of movement of the fluid transport configuration relative to the abutment surface, at least part of the path of movement having a non-zero component parallel to the planar surface; wherein the microneedles are arranged so that a leading one of the microneedles defines an effective area which is void of another of the microneedles, the effective area being defined as an area marked out by translating the base area of the leading microneedle, by the height of the leading microneedle, in a direction opposite to the non-zero component.

According to a further feature of the present invention, a spacing of the microneedles in the direction is at least the square root of 2 times a closest neighbor spacing.

According to the teachings of the present invention there is also provided a microneedle device for transporting fluid across a biological barrier, the device comprising: (a) a substrate defining a substantially planar surface; and (b) a plurality of microneedles projecting from the surface, each of the microneedles having a penetrating tip, a cutting edge, a base area and a height, each of the microneedles having a base-to-tip vector defined as a vector from a centroid of the base area to a centroid of the penetrating tip, the microneedles being asymmetrical such that the base-to-tip vector is non-perpendicular to the surface, a direction parallel to a projection of the base-to-tip vector on to the planar surface being taken to define a penetration direction, the microneedles being arranged so that a leading one of the microneedles defines an effective area which is void of another of the microneedles, the effective area being defined as an area marked out by translating the base area of the leading microneedle, by the height of the leading microneedle, in a direction opposite to the penetration direction.

According to a further feature of the present invention, a spacing of the microneedles in the penetration direction is at least the square root of 2 times a closest neighbor spacing.

According to the teachings of the present invention there is also provided a microneedle device for transporting fluid through a surface of a biological barrier, the device comprising: (a) a fluid transport configuration including: (i) a substrate having a surface; and (ii) a plurality of microneedles projecting from the surface of the substrate, each of the microneedles having a penetrating tip and a cutting edge, the microneedles being arranged in a plurality of rows; (b) an abutment member having at least one abutment surface for abutting the biological barrier, the abutment member being mechanically connected to the fluid transport configuration; and a (c) displacement device operationally connected to the abutment member, the displacement device configured for generating a relative lateral sliding movement between the fluid transport configuration and the surface of the biological barrier, at least one of the fluid transport configuration and the abutment member being configured such that, a leading one of the rows of the microneedles contacts the biological barrier prior to a trailing one of the rows of the microneedles contacting the biological barrier.

According to a further feature of the present invention, the displacement device mechanically links the abutment member and the fluid transport configuration, the displacement device defining a path of movement of the fluid transport configuration relative to the abutment surface, at least part of the path of movement having a non-zero component parallel to the surface of the substrate.

According to a further feature of the present invention: (a) the abutment member is configured as a suction cup, the fluid transport configuration being disposed in the suction cup; and (b) the displacement device includes a suction arrangement in fluid connection with the suction cup, the suction arrangement being configured for generating suction for pulling the surface of the biological barrier into the suction cup thereby generating the relative lateral sliding movement between the fluid transport configuration and the surface of the biological barrier.

According to a further feature of the present invention: (a) the abutment surf lies on a first plane; (b) the surface of the substrate lies on a second plane; and (c) the first plane is oblique to the second plane.

According to a further feature of the present invention, the suction cup has an internal surface which is axis asymmetrical.

According to a further feature of the present invention, the suction cup includes a side trough in fluid connection with the suction arrangement, the suction arrangement and the side trough being configured such that, after the surface of the biological barrier has made contact with the microneedles, the biological barrier is pulled into the side trough thereby pulling the surface of the biological barrier across the surface of the substrate.

According to the teachings of the present invention there is also provided a microneedle device for transporting a fluid through a surface of a biological barrier, the device comprising: (a) a fluid transport configuration including: (i) a substrate having a surface; and (ii) a plurality of microneedles projecting from the surface; (b) an abutment member configured as a suction cup having at least one abutment surface for abutting the biological barrier, the fluid transport configuration being disposed in the suction cup; and (c) a displacement device including a suction arrangement in fluid connection with the suction cup, the suction arrangement including a suction plunger, the suction arrangement being configured for generating suction for pulling the surface of the biological barrier into the suction cup with a single one-directional movement of the suction plunger to a retracted position in the suction arrangement.

According to a further feature of the present invention, each of the microneedles has a cutting edge and a penetrating tip.

According to a further feature of the present invention, the suction arrangement includes a locking mechanism for retaining the suction plunger in the retracted position.

According to a further feature of the present invention, there is also provided a fluid injection plunger arrangement having a fluid plunger, the fluid injection plunger arrangement being in fluid connection with the fluid transport configuration, such that depressing the fluid plunger delivers the fluid via the fluid transport configuration.

According to a further feature of the present invention, the fluid injection plunger arrangement is disposed within the suction arrangement.

According to a further feature of the present invention, there is also provided a priming port in fluid connection with the fluid injection plunger arrangement, the priming port being configured for providing a fluid connection between an external supply of the fluid and the fluid injection plunger arrangement during filling of the fluid injection plunger arrangement with the fluid.

According to a further feature of the present invention, the fluid injection plunger arrangement has a movement restriction arrangement configured to prevent negative pressure within the suction cup from pulling down the fluid plunger.

According to the teachings of the present invention there is also provided a microneedle device for transporting fluid through a surface of a biological barrier, the device comprising: (a) a fluid transport configuration including: (i) a substrate having a surface; and (ii) a plurality of microneedles projecting from the surface of the substrate, each of the microneedles having a penetrating tip and a cutting edge; (b) an abutment member configured as a suction cup, the fluid transport configuration being disposed in the suction cup; and (c) a displacement device including a suction arrangement in fluid connection with the suction cup, the suction arrangement being configured for generating suction for pulling the surface of the biological barrier into the suction cup thereby generating a relative lateral sliding movement between the fluid transport configuration and the surface of the biological barrier.

According to the teachings of the present invention there is also provided a microneedle device for transporting fluid through a surface of a biological barrier, the device comprising: (a) a fluid transport configuration including: a (i) substrate having a surface; and (ii) a plurality of microneedles projecting from the surface; (b) an abutment member having at least one abutment surface for abutting the biological barrier; and (c) a displacement device mechanically connected to the abutment member and the fluid transport configuration, the displacement device defining a rotational path of movement of the fluid transport configuration relative to the abutment member.

According to a further feature of the present invention, the rotational path of movement is about an axis substantially parallel to the initial orientation of the surface of the biological barrier.

According to a further feature of the present invention, each of the microneedles has a cutting edge and a penetrating tip.

### BRIEF DESCRIPTION OF THE DRAWINGS

The invention is herein described, by way of example only, with reference to the accompanying drawings, wherein:

FIG. 1a is an isometric view of a microneedle that is constructed and operable in accordance with the prior art;

FIG. 1b is another isometric view of the microneedle of FIG. 1a;

FIG. 2a is a schematic isometric view of a pyramidal microneedle that is constructed and operable in accordance with the prior art;

FIG. 2b is a schematic plan view of the microneedle of FIG. 3a;

FIG. 2c is a schematic view of a base-to-tip vector of the microneedle of FIG. 2a;

FIG. 3a is a schematic view tubular microneedle that is constructed and operable in accordance with the prior art;

FIG. 3b is a schematic plan view of the microneedle of FIG. 3a;

FIG. 3c is a schematic view of a base-to-tip vector of the microneedle of FIG. 3a;

FIG. 4 is a schematic isometric view of a fluid transport configuration that is constructed and operable in accordance with a preferred embodiment of the present invention;

FIG. 5 is a schematic side view of a fluid transport configuration that is constructed and operable in accordance with an alternate embodiment of the present invention;

FIG. 6 is a schematic side view of a microneedle device including the fluid transport configuration of FIG. 4;

FIG. 7 is a schematic side view of a microneedle device including the fluid transport configuration of FIG. 5;

FIG. 8a is an axial sectional view of a microneedle device including the fluid transport configuration of FIG. 5;

FIG. 8b is an exploded view of the microneedle device of FIG. 8a;

FIG. 8c is a view of the microneedle device of FIG. 8a after fluid is drawn therein;

FIG. 8d is a view of the microneedle device of FIG. 8c after the biological barrier pulled therein;

FIG. 8e is an expanded view of the lower section of the microneedle device of FIG. 8d;

FIG. 8f is a view of the microneedle device of FIG. 8d after the fluid is delivered through the surface of the biological barrier;

FIG. 9 is a cross-sectional view of a microneedle device employing the concept of the fluid transport configuration of FIG. 5;

FIG. 10 is a cross-sectional view of a microneedle device including the fluid transport configuration of FIG. 4;

FIG. 11a is an isometric view of a microneedle device which is constructed and operable in accordance with a preferred embodiment of the present invention;

FIG. 11b is a plan view of the device of FIG. 11a;

FIG. 11c is a cross-sectional view through line A-A of FIG. 11b prior to use of the device;

FIG. 11d is a cross-sectional view through line B-B of FIG. 11b prior to use of the device;

FIG. 11e is a cross-sectional view through line A-A of FIG. 11b showing the device in an intermediate position;

FIG. 11f is a cross-sectional view through line A-A of FIG. 11b showing the device in a final position;

FIG. 11g is an expanded view of region B of FIG. 11c showing the device prior to insertion into the biological barrier;

FIG. 11h is an expanded view of region C of FIG. 11f showing the device inserted into the biological barrier;

FIG. 11i is a partial cross-sectional view of a microneedle device prior to insertion into the biological barrier having microneedles facing the opposite direction to that of the device of FIG. 11a; and

FIG. 11j is a view of the microneedle device of FIG. 11i inserted into the biological barrier.

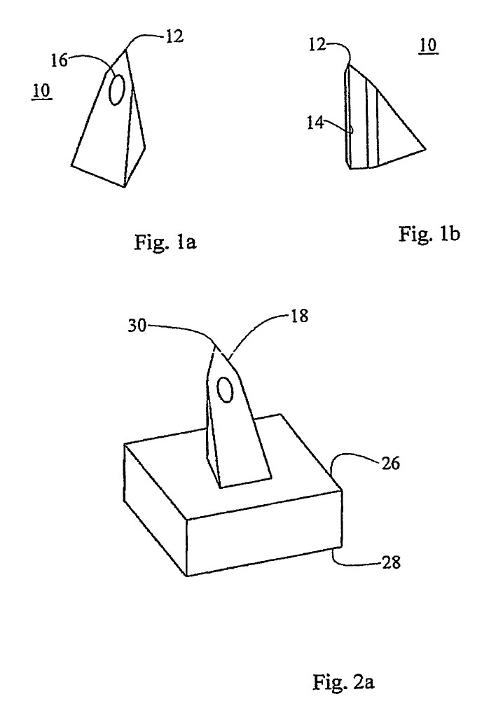

### DESCRIPTION OF THE PREFERRED EMBODIMENTS

The present invention is a microneedle device and method of operation thereof.

The principles and operation of a microneedle device according to the present invention may be better understood with reference to the drawings and the accompanying description.

As described hereinabove, WO 03/074102, co-assigned with the present application, teaches improved microneedle penetration devices using directional insertion, preferably using asymmetric microneedles, to enhance penetration of the biological barrier. It is explained in the aforementioned publication that the flexibility of the skin is particularly pronounced under out-of-plane deformations, allowing the skin to be locally depressed so as to conform to the external shape of the microneedles without allowing proper penetration. This effect seriously impedes, or even prevents, fluid transfer via the microneedles. However, the directional insertion device includes generating a displacement of the microneedle substrate surface relative to the biological barrier, the displacement having a non-zero component parallel to the surface of the substrate. In contrast to the out-of-plane flexibility of the biological barrier, the in-plane stretching capabilities of the skin are much more limited. These contrasting properties are familiar to us from everyday experience in which relatively blunt objects which do not pierce the skin on localized pressure readily cause scratches under sliding contact conditions. As a result of these properties, a penetration vector which includes a component parallel to the skin surface tends to be much more effective than direct pressure perpendicular to the skin.

Directional insertion represents a great improvement over other existing microneedle insertion devices. Nevertheless, it has been found that the penetration effect or the directional insertion device can be improved. Particularly, it has been found that the penetration and/or cutting effectiveness (if the microneedle has a cutting edge) of a leading microneedle in an array is reduced by a trailing microneedle in the same array, due to tension release created by the trailing needle on the biological barrier. The above problem is not limited to the first row of microneedles in an array, but to every row of microneedles in an array which has another row of microneedles trailing behind it.

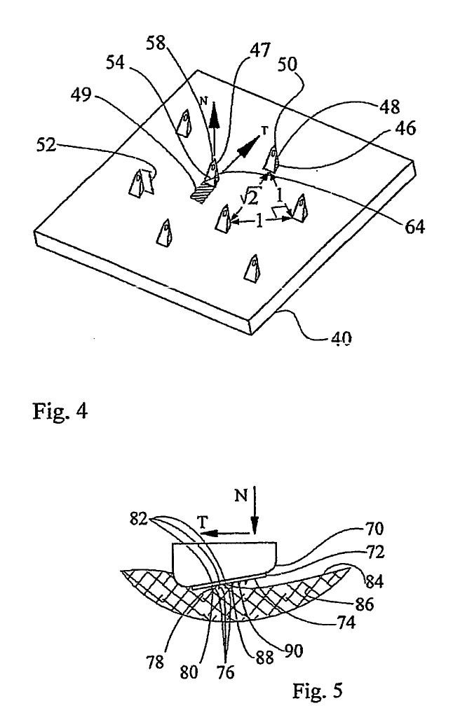

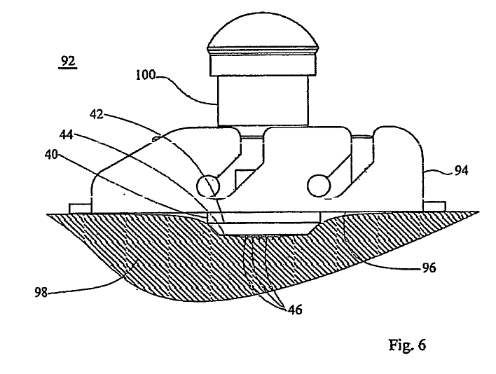

The above problem is removed or greatly reduced by either arranging the microneedles using a specific layout, as will be explained in more detail with reference to FIG. 4, and/or by ensuring that a leading row of microneedles makes contact with the biological barrier before a trailing row of microneedles makes contact with the biological barrier, as will be explained in more detail with reference to FIG. 5.

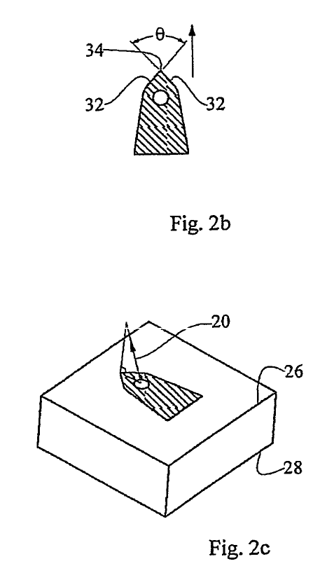

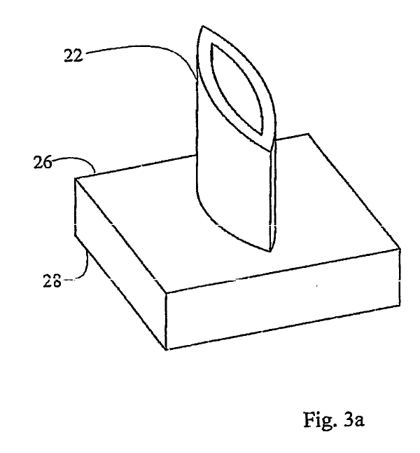

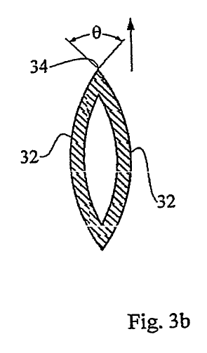

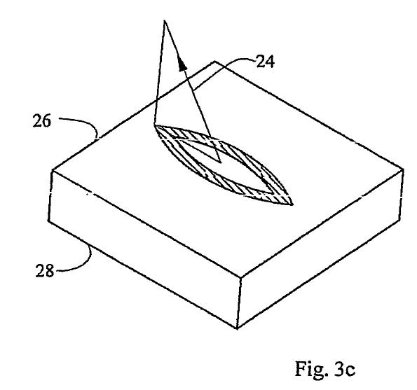

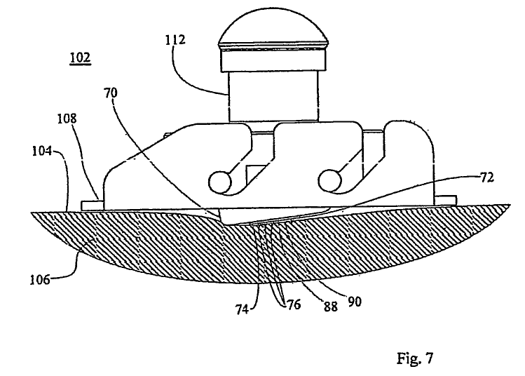

Reference is now made to FIG. 4, which is a schematic isometric view of a fluid transport configuration 40 that is constructed and operable in accordance with a preferred embodiment of the present invention. Fluid transport configuration 40 includes a substrate 42 defining a substantially planar surface 44. Fluid transport configuration 40 also includes a plurality of microneedles 46 projecting from surface 44. Each microneedle 46 is a micropyramid having a cutting edge 48, a penetrating tip 50, a height 52 and a base area 54. Height 52 is the height of the microneedle as measured perpendicularly from surface 44. Height 52 is typically the shortest distance from penetrating tip 50 to surface 44. Each microneedle 46 has a base-to-tip vector defined as a vector from a centroid of base area 54 to a centroid of penetrating tip 50. The term “base-to-tip” vector has been defined herein above with reference to FIGS. 2c and 3c. Microneedles 46 are asymmetrical such that their base-to-tip vector is non-perpendicular to surface 44. A direction parallel to a projection of the base-to-tip vector on to surface 44 is taken to define a penetration direction, T. It will be appreciated by those ordinarily skilled in the art that fluid transport configuration 40 is generally included as part of a directional insertion device (not shown) which defines a relative path of motion of fluid transport configuration 40 such that the path of motion has a component, N, perpendicular to surface 44 and a component parallel to surface 44 in penetration direction, T also termed a “sliding direction” of microneedles 46. Therefore, the directional insertion device generates a relative lateral sliding movement between fluid transport configuration 40 and a surface of a biological barrier. The term “relative lateral sliding movement” is defined to include either movement of fluid transport configuration 40 across the surface of the biological barrier or movement of the surface of the biological barrier across a stationary fluid transport configuration 40 or a combination of both. Suitable directional insertion devices are described below with reference to FIGS. 6 to 11i.

Microneedles 46 are arranged in rows perpendicular to penetration direction, T. In order to reduce or eliminate the pulling effect of a trailing microneedle on a leading microneedle, microneedles 46 are arranged so that a leading microneedle 47 defines an effective area 49 behind leading microneedle 47 which is void of another microneedle. Area 49 is defined by the area marked out by translating base area 54 of leading microneedle 47 by height 52 of leading microneedle 47 in a direction opposite to penetration direction, T.

Additionally, in order to maximize the microneedles density, while still keeping to the abovementioned spacing criteria, the microneedle spacing in penetration direction, T is at least the square root of 2, times the closest neighbor spacing. The term “spacing” is defined as the distance between centroids of the base areas of the microneedles.

It will be appreciated by those ordinarily skilled in the art that many layout patterns are possible within the above guidelines, as long as the microneedles are spaced so that an “effective area” behind a leading microneedle is not occupied by a trailing microneedle.

Reference is now made to FIG. 5, which is a schematic side view of a fluid transport configuration 70 that is constructed and operable in accordance with an alternate embodiment of the present invention. Fluid transport configuration 70 includes a substrate 72 having a surface 74. Fluid transport configuration 70 also includes a plurality of microneedles 76 projecting from surface 74. Each microneedle 76 is a micropyramid having a cutting edge 78 and a penetrating tip 80. Microneedles 76 are arranged in a plurality of rows 82. Fluid transport configuration 70 is generally included as part of a of a directional insertion device (not shown). Suitable directional insertion devices are described with reference to FIGS. 7 to 11i. The directional insertion device defines a path of motion of fluid transport configuration 70 such that the path of motion has a component, N, perpendicular to a surface 84 of a biological barrier 86 and a component parallel to surface 84 in penetration direction, T. Therefore, the directional insertion device generates a relative lateral sliding movement between fluid transport configuration 70 and surface 84 of biological barrier 86. The term “relative lateral sliding movement” is defined to include either movement of fluid transport configuration 70 across surface 84 of biological barrier 86 or movement of surface 84 of biological barrier 86 across a stationary fluid transport configuration 70 or a combination of both. In order to reduce or eliminate the pulling effect of a trailing microneedle on a leading microneedle, the directional insertion device including fluid transport configuration 70 is configured such that a leading row 88 of microneedles 76 contacts surface 84 of biological barrier 86 prior to a trailing row 90 of microneedles 76 contacting surface 84 of biological barrier 86. The directional insertion device including fluid transport configuration 70 is preferably configured such that leading row 88 at least partially cuts into surface 84 prior to a trailing row 90 of microneedles 76 contacting surface 84 of biological barrier 86.

In accordance with a most preferred embodiment of the present invention, fluid transport configuration 70 incorporates the microneedle layout determined by the criteria described with reference to FIG. 4, above.

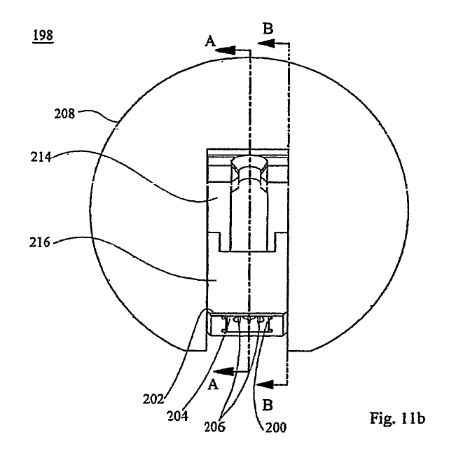

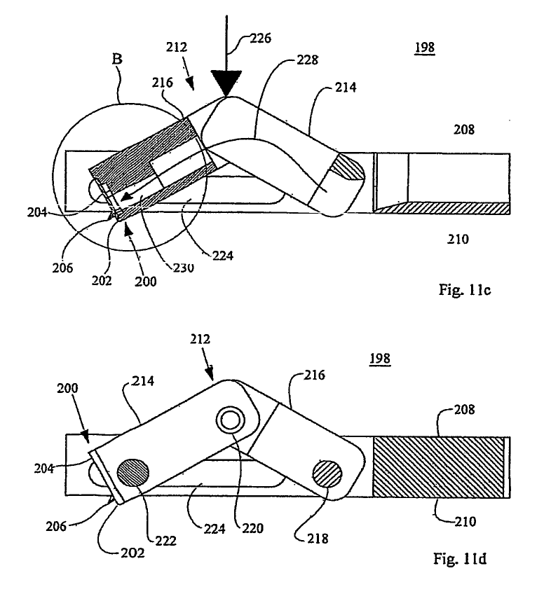

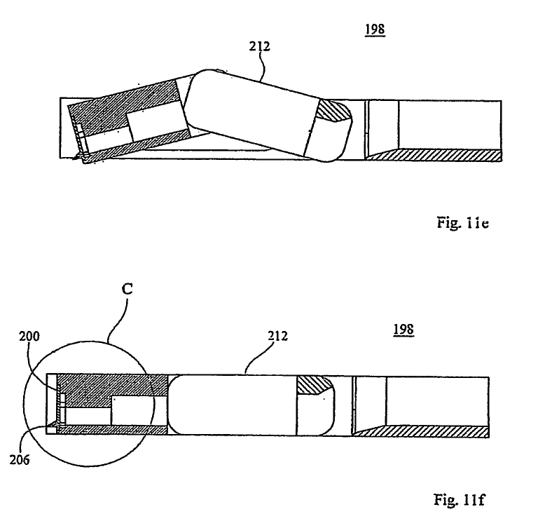

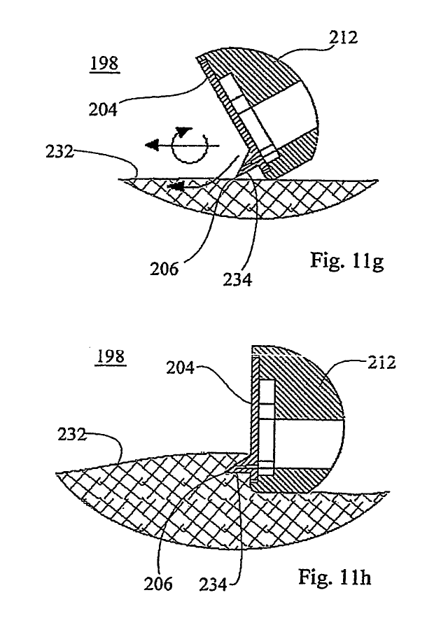

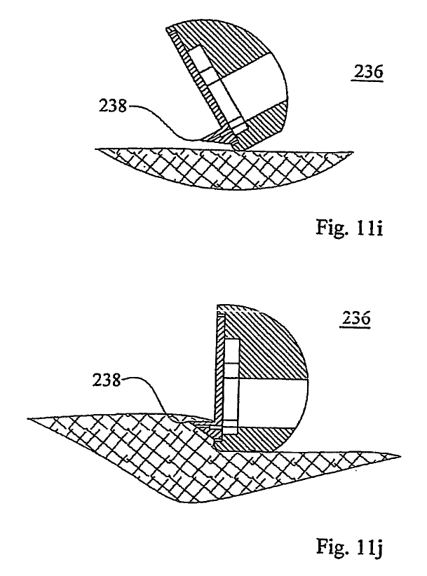

Reference is now made to FIG. 6, which is a schematic side view of a microneedle device 92 including fluid transport configuration 40 of FIG. 4. Microneedle device 92 operates substantially the same as the directional insertion devices taught with reference to WO 03/074102 except that the fluid transport configuration is the same as fluid transport configuration 40 and therefore the microneedle layout is determined by the criteria described with reference to FIG. 4, above. Microneedle device 92 includes an abutment member 94 having at least one abutment surface 96 for abutting a biological barrier 98. Microneedle device 92 also includes a displacement device 100 mechanically linking abutment member 94 and fluid transport configuration 40. Displacement device 100 defines a path of movement of fluid transport configuration 40 relative to abutment surface 96. Part of the path of movement has a non-zero component parallel to surface 44 of substrate 42 of fluid transport configuration 40. The penetration direction of microneedles 46 is defined by the non-zero component of the path of movement parallel to surface 44. As described above with reference to FIG. 4, the “penetration direction” or “sliding direction” of microneedles 46 is also defined by the base-to-tip vector of microneedles 46. However, it should be noted that if the penetration (or sliding) direction of the microneedles as defined by the directional insertion device is not the “natural” sliding direction of the microneedles (as defined by the geometry of the microneedles), then the penetration direction defined by the directional insertion device prevails as the operative definition of the penetration or sliding direction. An example of this is the embodiment of FIGS. 11a-h.

It is clear, that for effective microneedle penetration, the penetration direction defined by the base-to-tip vector of microneedles 46 is the same as the penetration direction defined by the path of movement of fluid transport configuration 40 as defined by displacement device 100.

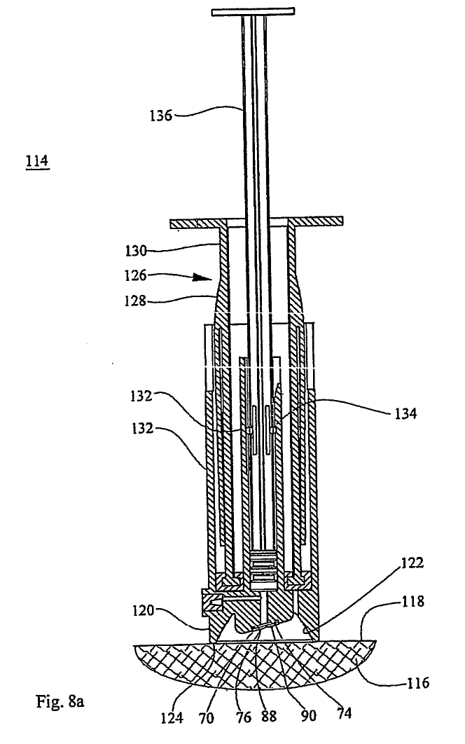

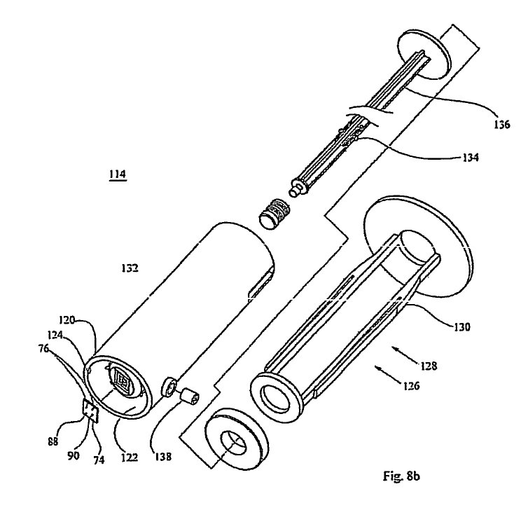

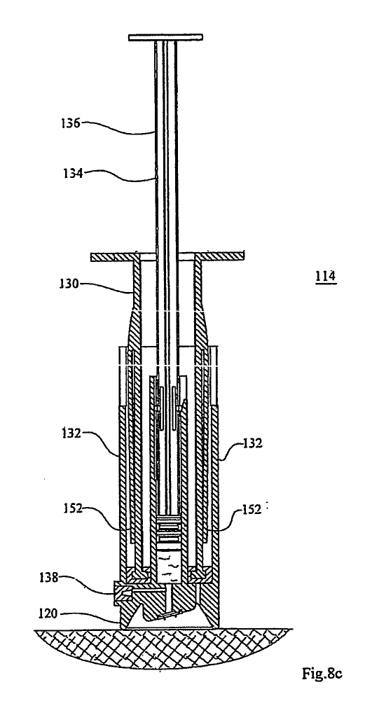

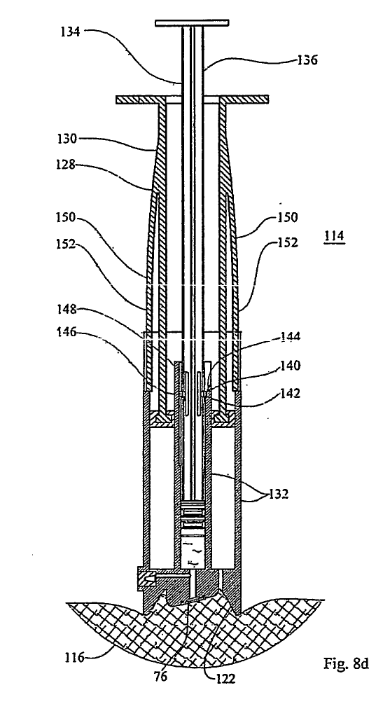

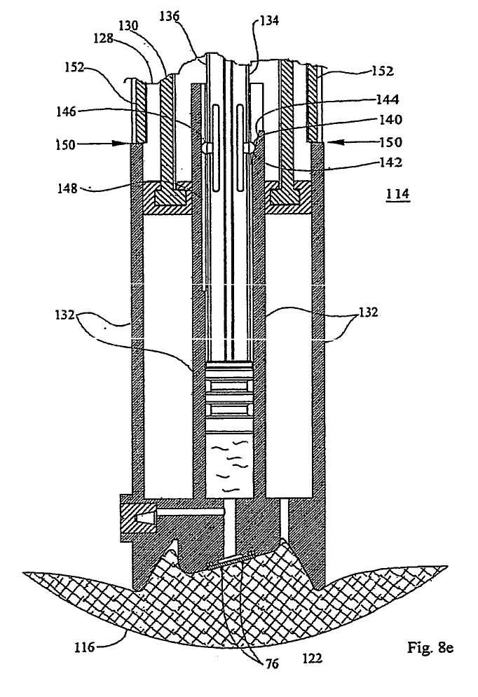

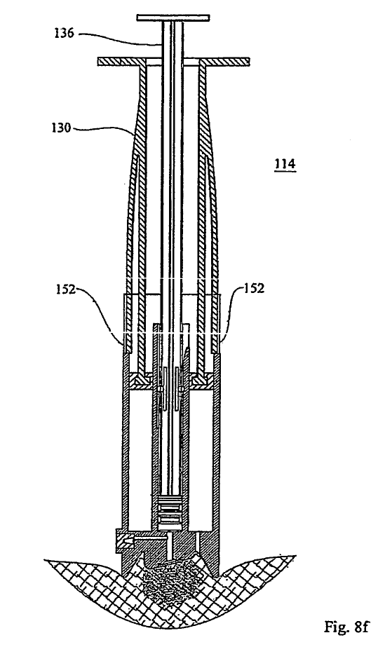

Reference is now made to FIG. 7, which is a schematic side view of a microneedle device 102 including fluid transport configuration 70 of FIG. 5. Microneedle device 102 is substantially the same as the directional insertion devices taught with reference to WO 03/074102 except for the differences described hereinbelow. Microneedle device 102 includes an abutment member 108 having at least one abutment surface 110 for abutting a surface 104 of a biological barrier 106. Microneedle device 102 includes a displacement device 112 mechanically linking abutment member 108 and fluid transport configuration 70. Displacement device 112 defines a path of movement of fluid transport configuration 70 relative to abutment surface 110. Part of the path of movement has a non-zero component parallel to surface 74 of substrate 72. Therefore, displacement device 112 generates a relative lateral sliding movement between fluid transport configuration 70 and surface 104 of biological barrier 106. Microneedle device 102 is configured so that leading row 88 of microneedles 76 contacts surface 104 of biological barrier 106 prior to trailing row 90 of microneedles 76 contacting surface 104 of biological barrier 106. This effect is typically achieved by slanting surface 74 of fluid transport configuration 70 with respect to abutment surface 110. The slant of surface 74 with respect to abutment surface 110 is typically between 5 and 25 degrees depending on the microneedle spacing which is typically between 500 and 700 microns between rows. Reference is now made to FIGS. 8a and 8b. FIG. 8a is an axial sectional view of a microneedle device 114 including fluid transport configuration 70 of FIG. 5. FIG. 8b is an exploded view of device 114 of FIG. 8a Device 114 is a suction device configured for bringing a surface 118 of a biological barrier 116 into contact with microneedles 76 of fluid transport configuration 70 so that leading row 88 of microneedles 76 contacts surface 118 of biological barrier 116 prior to trailing row 90 of microneedles 76 contacting surface 118 of biological barrier 116. Additionally, device 114 creates a lateral sliding motion between surface 118 of biological barrier 116 and microneedles 76 of fluid transport configuration 70, as will be described in more detail, below. Device 114 includes an abutment member 120 including a suction cup 122 having a continuous abutment surface 124. Fluid transport configuration 70 is disposed centrally in suction cup 122. Fluid transport configuration 70 is slanted with respect to abutment surface 124 such that abutment surface 124 lies of a first plane and surface 74 of fluid transport configuration 70 lies on a second plane, the first plane being oblique to the second plane. Device 114 includes a displacement device 126 including a suction arrangement 128 in fluid connection with suction cup 122. Suction arrangement 128 includes a suction plunger 130 disposed in a plunger housing 132. Plunger housing 132 is rigidly mechanically connected to abutment member 120. Suction arrangement 128 is configured for generating suction for pulling surface 118 of biological barrier 116 into suction cup 122 with a single one-directional movement of suction plunger 130 to a retracted position in suction arrangement 128, thereby generating a relative lateral sliding movement between microneedles 76 of fluid transport configuration 70 and surface 118 of biological barrier 116. The suction generated by suction arrangement 128 exerts a pulling force on biological barrier 116 so that biological barrier 116 is pulled evenly into suction cup 122. As surface 118 of biological barrier 116 makes contact with leading row 88 of microneedles 76, microneedles 76 anchor a region of surface 118 of biological barrier 116. The free portion of biological barrier 116 (in other words, the portion of biological barrier 116 not restricted by the anchoring effect) is pulled further into suction cup 122 thereby stretching surface 118 and creating a lateral sliding movement between microneedles 76 of leading row 88 as these microneedles 76 cut surface 118. Surface 118 is then anchored by the next row of microneedles 76 and the skin is pulled by the suction and stretched and the anchored microneedles 76 cut surface 118. This process continues until biological barrier 116 fills the cavity of suction cup 122 as shown best in FIG. 8d. Device 114 also includes a fluid injection plunger arrangement 134 having a fluid plunger 136. Fluid injection plunger arrangement 134 is disposed within suction arrangement 128 so that fluid injection plunger arrangement 134 and suction arrangement 128 share a common wall of plunger housing 132. Fluid injection plunger arrangement 134 and suction arrangement 128 form a coaxial arrangement. This coaxial arrangement has many advantages, including ease of use whereby suction of biological barrier 116 and injection of fluid into biological barrier 116 can be performed with the same hand. Fluid injection plunger arrangement 134 is in fluid connection with fluid transport configuration 70 such that depressing fluid plunger 136 delivers the fluid via microneedles 76 of fluid transport configuration 70. Priming of fluid injection plunger arrangement 134 is described in more detail with reference to FIG. 8c. Injection of the fluid is described with reference to FIG. 8f.

Reference is now made to FIG. 8c, which is a view of device 114 of FIG. 8a after the fluid is drawn therein. Device 114 also includes a priming port 138 disposed in the side of abutment member 120. A regular syringe having a prefixed dose of medication is brought into contact with priming port 138. Printing port 138 is configured for providing a fluid connection between the regular syringe (an external supply of the fluid) and fluid injection plunger arrangement 134 during filling of fluid injection plunger arrangement 134 with the fluid. Priming port 138 is in fluid connection with fluid injection plunger arrangement 134 such that retraction of fluid plunger 136 draws the fluid into fluid injection plunger arrangement 134 via priming port 138 from the regular syringe. However, it will be appreciated by those ordinarily skilled in the art that fluid injection plunger arrangement 134 can be filled by depressing on the plunger of the regular syringe. Priming port 138 includes a pierceable non-cored septum (not shown), which acts like a one way valve for preventing the fluid being forced through priming port 138 when fluid plunger 136 is depressed. Additionally, there is a one-way valve (not shown) disposed between fluid injection plunger arrangement 134 and fluid transport configuration 70 for preventing air being sucked into fluid injection plunger arrangement 134 via microneedles 76 when fluid is sucked into fluid injection plunger arrangement 134. Reference is now made to FIGS. 8d and 8e. FIG. 8d is a view of device 114 of FIG. 8c after biological barrier 116 is pulled therein. FIG. 8e is an expanded view of the lower section of device 114 of FIG. 8d. Fluid injection plunger arrangement 134 has a movement restriction arrangement 140 configured to prevent negative pressure within suction cup 122 from pulling fluid plunger 136 toward suction cup 122 and thereby dispensing the fluid before biological barrier 116 has been penetrated by microneedles 76. Movement restriction arrangement 140 includes a projection 142 projecting radially from fluid plunger 136. Once fluid plunger 136 has been retracted projection 142 engages into a recess 144 in plunger housing 132 thereby preventing negative pressure in suction cup 122 from pulling fluid plunger 136. Projection 142 is released from recess 144 by pushing on the handle of fluid plunger 136 with a force greater than a minimum threshold value. Due to the small diameter of fluid injection plunger arrangement 134, the required threshold force is achievable by every user. Fluid injection plunger arrangement 134 also includes another projection 146 projecting radially from fluid plunger 136. Projection 146 moves longitudinally within a slot 148 disposed within plunger housing 132 in order to prevent rotation of fluid plunger 136 within plunger housing 132. This rotation could neutralize the functionality of movement restriction arrangement 140. Additionally, projection 146 also ensure proper positioning of fluid plunger 136 in fluid injection plunger arrangement 134.

Suction arrangement 128 includes a locking mechanism 150 for retaining suction plunger 130 in a retracted position. Locking mechanism 150 includes two resilient arms 152. Resilient arms 152 are stored within plunger housing 132 while suction plunger 130 is depressed (best seen in FIG. 8c). When suction plunger 130 is retracted, resilient arms 152 are released from plunger housing 132 so that resilient arms 152 expand. Suction plunger 130 cannot be pulled into plunger housing 132 as resilient arms 152 rest on the top surface of plunger housing 132 thereby preventing downward movement of suction plunger 130. Locking mechanism 150 also controls the suction level required for optimal operation of device 114. Due to effects of fatigue in plastics, resilient arms 152 are kept under low (below 25% of yield) stress during shelf life to maintain their flexibility.

Reference is now made to FIG. 8f, which is a view of device 114 of FIG. 8d after the fluid is delivered through surface 118 of biological barrier 116. The fluid is delivered by depressing fluid plunger 136. After injection of the fluid, resilient arms 152 are compressed thereby allowing suction plunger 130 to be depressed for releasing the suction on biological barrier 116.

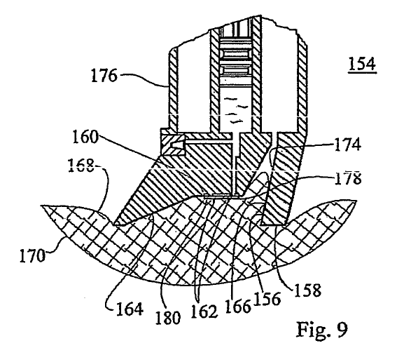

Reference is now made to FIG. 9, which is a cross-sectional view of a lower section of a microneedle device 154 employing the concept of fluid transport configuration 70 of FIG. 5. Device 154 is substantially the same as device 114 of FIGS. 8a-f except for the following differences. Device 154 has a suction cup 156 which has an abutment surface 158. Device also has a suction arrangement 176. Device 154 has a fluid transport configuration 160 including a substrate 178 having a surface 180. Surface 180 has a plurality of microneedles 162 disposed thereon. Each microneedle 162 includes a penetrating tip and a cutting edge. Suction cup 156 has an internal surface which is axis asymmetrical. The term “axis asymmetrical” is defined with reference to suction cup 156 not having an axis of symmetry. The embodiment of suction cup 156 shows an asymmetric cup formed by configuring the slant of the interior walls of suction cup 156 to have different gradients, one part 164 has a shallow gradient and one part 166 has a steep gradient. However, it will be appreciated by those ordinarily skilled in the art that axis asymmetry can be achieved in other ways, for example, but not limited to forming the abutment surface area as a non-circular shape such as, egg shaped. The axis asymmetry of suction cup 156 ensures that the pulling force on a surface 168 of a biological barrier 170 is uneven. Therefore, when biological barrier 170 is pulled in to suction cup 156 via the suction, a leading row of microneedles 162 contacts surface 168 before a trailing row of microneedles 162 contacts surface 168. Additionally, the anchoring and stretching effects of biological barrier 170 as described with reference to device 114 also occur with device 154. Parenthetically, suction cup 122 of device 114 also has an axis asymmetrical suction cup 122 caused by slating fluid transport configuration 70. Nevertheless, biological barrier 116 is pulled evenly by device 114 until it makes contact with fluid transport configuration 70 as the lower portion of suction cup 122 is symmetrical.

Suction cup 156 also includes a side trough 174 in fluid connection with suction arrangement 176. Suction arrangement 176 and side trough 174 are configured so that suction arrangement 176 pulls biological barrier 170 via side trough 174. Therefore, after surface 168 of biological barrier 170 has made contact with microneedles 162, biological barrier 170 is pulled into side trough 174 thereby pulling surface 168 of biological barrier 170 across surface 180 of substrate 178.

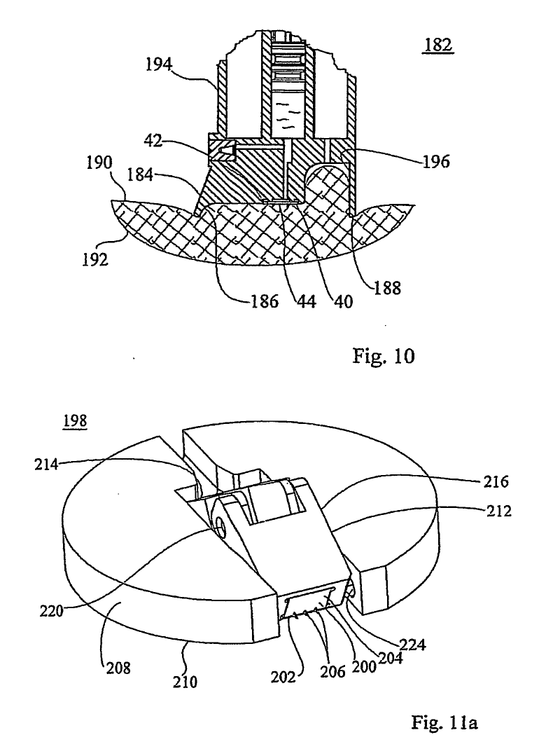

Reference is now made to FIG. 10, which is a cross-sectional view of a lower section of a microneedle device 182 including fluid transport configuration 40 of FIG. 4. Device 182 includes an abutment member 184 including a suction cup 186 having an abutment surface 188 for abutting a surface 190 of a biological barrier 192. Fluid transport configuration 40 is disposed in suction cup 186 so that the surface of fluid transport configuration 40 lies on a plane which is parallel to a plane defined by abutment surface 188. Suction cup 186 has a side trough 196. Device 182 includes a suction arrangement 194 in fluid connection with side trough 196 of suction cup 186. Suction arrangement 194 generates suction for pulling surface 190 of biological barrier 192 into suction cup 186. Suction arrangement 194 and side trough 196 are configured so that suction arrangement 194 pulls biological barrier 192 via side trough 196. Therefore, after surface 190 of biological barrier 192 has made contact with the microneedles of fluid transport configuration 40, biological barrier 192 is pulled into side trough 196 thereby pulling surface 190 of biological barrier 192 across surface 44 of substrate 42 of fluid transport configuration 40 in a sliding direction of microneedles 46. It should be noted that to enable consistent use of terminology, even though microneedles 46 are stationary, the sliding direction is defined with respect to microneedles 46 and not the sliding direction of surface 190 of biological barrier 192.

Reference is now made to FIGS. 11a-11d. FIG. 11a is an isometric view of a microneedle device 198 which is constructed and operable in accordance with a preferred embodiment of the present invention. FIG. 11b is a plan view of device 198 of FIG. 11a. FIG. 11c is a cross-sectional view through line A-A of FIG. 11b prior to use of device 198. FIG. 11d is a cross-sectional view through line B-B of FIG. 11b prior to use of device 198; Device 198 is for transporting a fluid through a surface of a biological barrier. Device 198 is designed for continuous delivery of fluid or where it is impossible to maintain suction of the biological barrier for a long time. By way of introduction, pressure below the surface of the biological barrier, mainly due to the fluid being injected, tries to eject the microneedles from the biological barrier. This problem is more pronounced for shorter microneedles, and pyramidal microneedles, in particular. Device 198 reduces the problems associated with this below surface pressure, by ensuring that the microneedles are inserted at an inclined angle to the normal surface of the biological barrier, as will be described below. Therefore, the pressure below the surface of the biological barrier is effectively neutralized. Device 198 includes a fluid transport configuration 200 including a substrate 202 having a surface 204. Fluid transport configuration 200 also includes a plurality of microneedles 206 projecting from surface 204. Each microneedles 206 has a cutting edge and a penetrating tip. Device 198 includes an abutment member 208 having an abutment surface 210 for abutting the biological barrier. Abutment surface 210 is typically attached to the biological barrier using a suitable adhesive or clamping device (not shown). Adhesion can be achieved by the use of a wide range of adhesives or adhesive tapes which are designed for use in medical applications, as are well known in the-art. Most preferably, abutment surface 210 substantially encircles fluid transport configuration 200 on three sides. This creates a convex shaped pocket of the biological barrier. The biological barrier needs to have some give so that device 198 can create a “step” in the surface of the biological barrier, as will be described in more detail with reference to FIG. 11h below. Device 198 includes a displacement device 212 mechanically linking abutment member 208 and fluid transport configuration 200. Displacement device 212 includes two blocks 214, 216. Block 214 is mechanically connected by a hinge 218 to abutment member 208. Block 214 is mechanically connected by a hinge 220 to one end of block 216. Fluid transport configuration 200 is disposed on the other end of block 216. Block 216 includes two projections 222 projecting from the side of block 216. Projections 222 are disposed close to the end of block 216 having fluid transport configuration 200 thereon. Abutment member 208 includes two slots 224. Slots 224 extend almost parallel to a plane lying on abutment surface 210. Projections 222 are configured for sliding along slots 224. The degree of parallelism of slots 224 with the plane of abutment surface 210 is used to control how fluid transport configuration 200 approaches the skin. In use, the joint between block 214 and block 216 is depressed. As the movement of displacement device 212 is restricted by hinges 218, 220 and guided by projections 222 moving along slots 224, fluid transport configuration 200 moves through a rotational and linear path. Therefore, displacement device 212 defines a rotational path of movement of fluid transport configuration 200 relative to abutment member 208 about an axis substantially parallel to the initial orientation of the surface of the biological barrier. The term “rotational path” is defined herein to include the possibility of linear motion with rotation motion. The term “substantially” parallel is defined as within 30 to 60-degrees of the initial orientation of the surface of the biological barrier. The term “initial orientation of the surface” is defined as the initial orientation of the surface of the biological barrier before the surface of the barrier is moved or stretched or flexed by device 198.

Reference is now made to FIG. 11c. A single push of displacement device 212 in the direction of an arrow 226, moves fluid transport configuration 200 through a rotation path. Displacement device 212 also includes a connection 230 to a reservoir (not shown) for storing the fluid for injecting. Connection 230 is typically a tube or any other common connection such as a luer connector. An arrow 228 depicts the direction of flow of the fluid through displacement device 212 into fluid transport configuration 200. The fluid is typically driven by an infusion pump. Reference is now made to FIG. 11e, which is a cross-sectional view through line A-A of FIG. 11b showing device 198 in an intermediate position of displacement device 212.

Reference is now made to FIG. 11f, which is a cross-sectional view through line A-A of FIG. 11b showing device 198 in a final position FIG. 11f shows the in-use position of device 198 where the fluid is injected through microneedles 206 of fluid transport configuration 200. Displacement device 212 is self-locking due to the geometry of displacement device 212. Additionally, device 198 is a low-profile device making it suitable for long-term fluid-transfer use.

Reference is now made to FIG. 11g and 11h FIG. 11g is an expanded view of region B of FIG. 11c showing device 198 prior to insertion into a biological barrier 232. FIG. 11h is an expanded view of region C of FIG. 11f showing the device 198 inserted into biological barrier 232. Microneedles 206 anchor the surface of biological barrier 232 as displacement device 212 starts to rotate (FIG. 11g). Displacement device 212 rotates creating a “step” in biological barrier 232. Microneedles 206 penetrate into the vertical surface of the “step” FIG. 11h). Microneedles 206 are disposed on surface 204 so that a cutting edge 234 of microneedles 206 is facing into the surface of biological barrier 232. The direction that cutting edge 234 faces affects two factors. First, the effect of pressure of the biological barrier trying to eject the microneedles. Second, fluid leakage along the microneedles sloping sides. The above embodiment has cutting edge 234 facing into the surface of biological barrier 232 thereby reducing fluid leakage.

Reference is now made to FIGS. 11i and 11j. FIG. 11i is a partial cross-sectional view of a microneedle device 236 prior to insertion into the biological barrier having microneedles 238 facing the opposite direction to that of device 198 of FIG. 11a FIG. 11j is a view of device 236 of FIG. 11i inserted into the biological barrier. Each microneedle 238 has a cutting edge 240. Microneedles 238 are disposed so that the cutting edge faces toward the surface of the biological barrier thereby canceling the effect of pressure acting as an ejector of the microneedles.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art which would occur to persons skilled in the art upon reading the foregoing description.

What is claimed is:

1. A microneedle device for transporting fluid through a surface of a biological barrier, the device comprising:
(a) a fluid transport configuration including:
(i) a substrate having a substantially planar surface; and
(ii) a plurality of microneedles projecting from said planar surface, each of said microneedles having a cutting edge, a penetrating tip, a base area and a height;
(b) an abutment member having at least one abutment surface for abutting the biological barrier, said abutment member being mechanically connected to said fluid transport configuration; and
(c) a displacement device operationally connected to said abutment member, said displacement device configured for generating a relative lateral sliding movement between the surface of the biological barrier and said fluid transport configuration in a sliding direction of said microneedles,
wherein said microneedles are arranged so that a leading one of said microneedles defines an effective area which is void of another of said microneedles, said effective area being defined as an area marked out by translating said base area of said leading microneedle, by said height of said leading microneedle, in a direction opposite to said sliding direction.

2. The device of claim 1 wherein a spacing of said microneedles in said sliding direction is at least the square root of 2 times a closest neighbor spacing.

3. The device of claim 1 wherein:
(a) said abutment member is configured as a suction cup, said fluid transport configuration being disposed in said suction cup; and
(b) said displacement device includes a suction arrangement in fluid connection with said suction cup, said suction arrangement being configured for generating suction for pulling the surface of the biological barrier into said suction cup, said suction cup and said fluid transport configuration being configured such that the surface of the biological barrier slides across said planar surface in said sliding direction.

4. The device of claim 3, wherein:
(a) said abutment surface lies on a first plane;
(b) said surface of said substrate lies on a second plane; and
(c) said first plane is oblique to said second plane.

5. The device of claim 3, wherein said suction cup has an internal surface which is axis asymmetrical.

6. The device of claim 3, wherein said suction cup includes a side trough in fluid connection with said suction arrangement, said suction arrangement and said side trough being configured such that, after the surface of the biological barrier has made contact with said microneedles, the biological barrier is pulled into said side trough thereby pulling the surface of the biological barrier across said surface of said substrate.

7. The device of claim 3, wherein said displacement device mechanically links said abutment member and said fluid transport configuration, said displacement device defining a path of movement of said fluid transport configuration relative to said abutment surface, at least part of said path of movement having a non-zero component parallel to said surface of said substrate.

8. The device of claim 3, wherein said suction arrangement includes a suction plunger, said suction arrangement being configured for generating suction for pulling the surface of the biological barrier into said suction cup with a single one-directional movement of said suction plunger to a retracted position in said suction arrangement.

9. The device of claim 8, wherein said suction arrangement includes a locking mechanism for retaining said suction plunger in said retracted position.

10. The device of claim 3, further comprising a fluid injection plunger arrangement having a fluid plunger, said fluid injection plunger arrangement being in fluid connection with said fluid transport configuration, such that depressing said fluid plunger delivers the fluid via said fluid transport configuration.

11. The device of claim 10, wherein said fluid injection plunger arrangement is disposed within said suction arrangement.

12. The device of claim 10, further comprising a priming port in fluid connection with said fluid injection plunger arrangement, said priming port being configured for providing a fluid connection between an external supply of the fluid and said fluid injection plunger arrangement during filling of said fluid injection plunger arrangement with the fluid.

13. The device of claim 10, wherein said fluid injection plunger arrangement has a movement restriction arrangement configured to prevent negative pressure within said suction cup from pulling down said fluid plunger.

14. The device of claim 1, wherein at least one of said fluid transport configuration and said abutment member are configured such that, a leading one of said rows of said microneedles contacts the biological barrier prior to a trailing one of said rows of said microneedles contacting the biological barrier.

15. The device of claim 1, wherein said displacement device is mechanically connected to said abutment member and said fluid transport configuration, said displacement device defining a rotational path of movement of said fluid transport configuration relative to said abutment member.

16. The device of claim 15, wherein said rotational path of movement is about an axis substantially parallel to the initial orientation of the surface of the biological barrier.

17. A microneedle device for transporting fluid across a biological barrier, the device comprising:
(a) a fluid transport configuration including:
(i) a substrate having a substantially planar surface; and
(ii) a plurality of microneedles projecting from said surface, each of said microneedles having a penetrating tip, a cutting edge, a base area and a height;
(b) an abutment member having at least one abutment surface for abutting the biological barrier; and
(c) a displacement device mechanically linking said abutment member and said fluid transport configuration, said displacement device defining a path of movement of said fluid transport configuration relative to said abutment surface, at least part of said path of movement having a non-zero component parallel to said planar surface;
wherein said microneedles are arranged so that a leading one of said microneedles defines an effective area which is void of another of said microneedles, said effective area being defined as an area marked out by translating said base area of said leading microneedle, by said height of said leading microneedle, in a direction opposite to said non-zero component.

18. The device of claim 17, wherein a spacing of said microneedles in said direction is at least the square root of 2 times a closest neighbor spacing.

19. A microneedle device for transporting fluid across a biological barrier, the device comprising: a substrate defining a substantially planar surface; and a plurality of microneedles projecting from said surface, each of said microneedles having a penetrating tip, a cutting edge, a base area and a height, each of said microneedles having a base-to-tip vector defined as a vector from a centroid of said base area to a centroid of said penetrating tip, said microneedles being asymmetrical such that said base-to-tip vector is non-perpendicular to said surface, a direction parallel to a projection of said base-to-tip vector on to said planar surface being taken to define a penetration direction, said microneedles being arranged so that a leading one of said microneedles defines an effective area which is void of another of said microneedles, said effective area being defined as an area marked out by translating said base area of said leading microneedle, by said height of said leading microneedle, in a direction opposite to said penetration direction.

20. The device of claim 19, wherein a spacing of said microneedles in said penetration direction is at least the square root of 2 times a closest neighbor spacing.

21. A microneedle device for transporting fluid through a surface of a biological barrier, the device comprising:
(a) a fluid transport configuration including:
(i) a substrate having a surface; and
(ii) a plurality of microneedles projecting from said surface of said substrate, each of said microneedles having a penetrating tip and a cutting edge, said microneedles being arranged in a plurality of rows;
(b) an abutment member having at least one abutment surface for abutting the biological barrier, said abutment member being mechanically connected to said fluid transport configuration; and
(c) a displacement device operationally connected to said abutment member, said displacement device configured for generating a relative lateral sliding movement between said fluid transport configuration and the surface of the biological barrier, at least one of said fluid transport configuration and said abutment member being configured such that, a leading one of said rows of said microneedles contacts the biological barrier prior to a trailing one of said rows of said microneedles contacting the biological barrier.

22. The device of claim 21, wherein said displacement device mechanically links said abutment member and said fluid transport configuration, said displacement device defining a path of movement of said fluid transport configuration relative to said abutment surface, at least part of said path of movement having a non-zero component parallel to said surface of said substrate.

23. The device of claim 21, wherein:
(a) said abutment member is configured as a suction cup, said fluid transport configuration being disposed in said suction cup; and
(b) said displacement device includes a suction arrangement in fluid connection with said suction cup, said suction arrangement being configured for generating suction for pulling the surface of the biological barrier into said suction cup thereby generating said relative lateral sliding movement between said fluid transport configuration and the surface of the biological barrier.

24. The device of claim 23, wherein:
(a) said abutment surface lies on a first plane;
(b) said surface of said substrate lies on a second plane; and
(c) said first plane is oblique to said second plane.

25. The device of claim 23, wherein said suction cup has an internal surface which is axis asymmetrical.

26. The device of claim 25, wherein said suction cup includes a side trough in fluid connection with said suction arrangement, said suction arrangement and said side trough being configured such that, after the surface of the biological barrier has made contact with said microneedles, the biological barrier is pulled into said side trough thereby pulling the surface of the biological barrier across said surface of said substrate.

27. A microneedle device for transporting a fluid through a surface of a biological barrier, the device comprising:
(a) a fluid transport configuration including:
(i) a substrate having a surface; and
(ii) a plurality of microneedles projecting from said surface;
(b) an abutment member configured as a suction cup having at least one abutment surface for abutting the biological barrier, said fluid transport configuration being disposed in said suction cup; and
(c) a displacement device including a suction arrangement in fluid connection with said suction cup, said suction arrangement including a suction plunger, said suction arrangement being configured for generating suction for pulling the surface of the biological barrier into said suction cup with a single one-directional movement of said suction plunger to a retracted position in said suction arrangement.

28. The device of claim 27, wherein each of said microneedles has a cutting edge and a penetrating tip.

29. The device of claim 27, wherein said suction arrangement includes a locking mechanism for retaining said suction plunger in said retracted position.

30. The device of claim 27, further comprising a fluid injection plunger arrangement having a fluid plunger, said fluid injection plunger arrangement being in fluid connection with said fluid transport configuration, such that depressing said fluid plunger delivers the fluid via said fluid transport configuration.

31. The device of claim 30, wherein said fluid injection plunger arrangement is disposed within said suction arrangement.

32. The device of claim 30, further comprising a priming port in fluid connection with said fluid injection plunger arrangement, said priming port being configured for providing a fluid connection between an external supply of the fluid and said fluid injection plunger arrangement during filling of said fluid injection plunger arrangement with the fluid.

33. The device of claim 30, wherein said fluid injection plunger arrangement has a movement restriction arrangement configured to prevent negative pressure within said suction cup from pulling down said fluid plunger.

34. A microneedle device for transporting fluid through a surface of a biological barrier, the device comprising:
(a) a fluid transport configuration including:
(i) a substrate having a surface; and
(ii) a plurality of microneedles projecting from said surface of said substrate, each of said microneedles having a penetrating tip and a cutting edge;
(b) an abutment member configured as a suction cup, said fluid transport configuration being disposed in said suction cup; and
(c) a displacement device including a suction arrangement in fluid connection with said suction cup, said suction arrangement being configured for generating suction for pulling the surface of the biological barrier into said suction cup thereby generating a relative lateral sliding movement between said fluid transport configuration and the surface of the biological barrier.

35. A microneedle device for transporting fluid through a surface of a biological barrier, the device comprising:
(a) a fluid transport configuration including:
(i) a substrate having a surface; and
(ii) a plurality of microneedles projecting from said surface;
(b) an abutment member having at least one abutment surface for abutting the biological barrier, and
(c) a displacement device mechanically connected to said abutment member and said fluid transport configuration, said displacement device defining a rotational path of movement of said fluid transport configuration relative to said abutment member.

36. The device of claim 35, wherein said rotational path of movement is about an axis substantially parallel to the initial orientation of the surface of the biological barrier.

37. The device of claim 35, wherein each of said microneedles has a cutting edge and a penetrating tip.
